(19) United States
(12) Patent Application Publication
(10) Pub. No.: US 2014/0356974 A1
(43) Pub. Date: Dec. 4, 2014

(54) COATINGS FOR MEASURING PH CHANGES

(71) Applicants: Wen-Jing Hu, Arlington, TX (US); Liping Tang, Arlington, TX (US)

(72) Inventors: Wen-Jing Hu, Arlington, TX (US); Liping Tang, Arlington, TX (US)

(21) Appl. No.: 13/905,520

(22) Filed: May 30, 2013

(51) Int. Cl. G01N 33/84; A61B 5/1455; A61K 49/00; G01N 31/22; A61B 5/145

(52) U.S. Cl. CPC G01N 33/84; G01N 31/221; A61B 5/14539; A61K 49/0032; A61B 5/1455
USPC 436/163; 422/547; 600/317

(57) ABSTRACT

A pH detectable coating. The pH detectable coating includes a pH insensitive fluorescent dye and a pH sensitive fluorescent dye. The pH insensitive fluorescent dye and the pH sensitive fluorescent dye are attached to a surface. The ratio of the fluorescent intensity of the pH insensitive fluorescent dye to the fluorescent intensity of the pH sensitive fluorescent dye varies according to the pH of an environment into which the surface is placed.

### CROSS-REFERENCE TO RELATED APPLICATIONS

Not applicable.

### BACKGROUND OF THE INVENTION

pH is an important environmental factor which influences the activities of organs, tissues, cells and many biological products, including proteins, enzymes, and molecular products. The changes of pH may also reflect the overall biological status. For example, inflammatory responses and infection may cause the reduction of tissue and blood pH, also called as tissue acidosis. Tissue acidosis is a hallmark of inflammatory diseases. Specifically, high hydrogen ion concentrations have been found in inflamed tissues (down to pH 5.4), in fracture-related hematomas (down to pH 4.7), in cardiac ischemia (down to pH 5.7) and in and around malignant tumors. The acidification within diseased tissue is likely caused by cell death and hyperactive inflammatory cells. Intracellular acidification has also been linked to cell death-apoptosis. An acidic pH environment has been shown to increase cell death and the production of inflammatory cytokines. Gastric mucosal pH has been used as a tool to evaluate the prognosis of critically ill patients. A pH greater than 4.5 has been linked to bacterial vaginosis, a disease of the vagina caused by bacteria.

Several pH sensitive fluorescent dyes have been synthesized to enhance or to reduce fluorescence intensity with pH changes, although only a few of these dyes maybe used to accurately detect acidic pH (pH 7.4). These low pH-sensitive dyes have been used to show the pH changes during endocytosis and exocytosis in vitro, and in inflamed tissue and tumors in vivo. However, due to the diffusion of these dyes in and out of cells and tissues at different rates, previous methods could not provide quantitative values of pH in different regions of normal and inflamed tissue. To solve the problem, ratiometric imaging probes have been developed to detect pH changes in solution. However, the probes have to be delivered to the cells via injection or oral uptake. The probes cannot stay at the implant site for a long time and may cause systemic side-effects.

Accordingly, there is a need in the art for a device which can measure pH in situ. Further, there is a need in the art for the device to be accurate regardless of depth. Additionally, there is need in the art for the device to be able to work with medical devices or implants.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a pH detectable coating. The pH detectable coating includes a pH insensitive fluorescent dye and a pH sensitive fluorescent dye. The pH insensitive fluorescent dye and the pH sensitive fluorescent dye are attached to a surface. The ratio of the fluorescent intensity of the pH insensitive fluorescent dye to the fluorescent intensity of the pH sensitive fluorescent dye varies according to the pH of an environment into which the surface is placed.

Another example embodiment includes a method of manufacturing a pH detectable coating. The method includes attaching a pH insensitive fluorescent dye to a surface and attaching a pH sensitive fluorescent dye to the surface. The ratio of the fluorescent intensity of the pH insensitive fluorescent dye to the fluorescent intensity of the pH sensitive fluorescent dye varies according to the pH of the environment into which the surface is placed.

Another example embodiment includes a method of measuring pH in situ. The method includes providing a pH detectable coating on a surface. The pH detectable coating includes a pH insensitive fluorescent dye and a pH sensitive fluorescent dye. The ratio of the fluorescent intensity of the pH insensitive fluorescent dye to the fluorescent intensity of the pH sensitive fluorescent dye varies according to the pH of an environment into which the surface is placed. The method also includes placing the surface in an environment and exposing the surface to electromagnetic radiation. The method further includes measuring a fluorescent ratio and converting the fluorescent ratio to a pH value.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of a pH detectable coating;

FIG. 2 illustrates various methods of attaching the pH detectable coating to a surface;

FIG. 3 is a flow chart illustrating a method of detecting in situ pH;

FIG. 4A illustrates an example of an emission spectrum of a pH detectable coating at various pH levels;

FIG. 4B illustrates an example of fluorescent intensity at various pH levels;

FIG. 4C illustrates a ratio of fluorescent intensity of a pH sensitive fluorescent dye to a pH insensitive fluorescent dye at various pH levels;

FIG. 5A illustrates an example of fluorescent intensities and ratio at different concentrations;

FIG. 5B illustrates an example of pH measurements at various depths;

FIG. 6A illustrates an example of a fluorescent ratio compared to pH microelectrode measurements;

FIG. 6B illustrates an example of the correlation between measured pH and pH as calculated from a pH detectable coating;

FIG. 7A illustrates an example of pH measurements at different pH levels of a dye coated polyurethane catheter;

FIG. 7B illustrates an example of fluorescent intensity and fluorescent ratio of various pH levels of a dye coated polyurethane catheter;

FIG. 7C illustrates an example of correlation between fluorescent ratio and pH

FIG. 8 illustrates an example of pH measurements at different depths of a dye coated polyurethane catheter;

FIG. 9A illustrates an example of fluorescent intensity of various implant materials with a pH detectable coating over time; and

FIG. 9B illustrates an example of fluorescent intensity change of various implant materials with a pH detectable coating at different in situ pH values.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

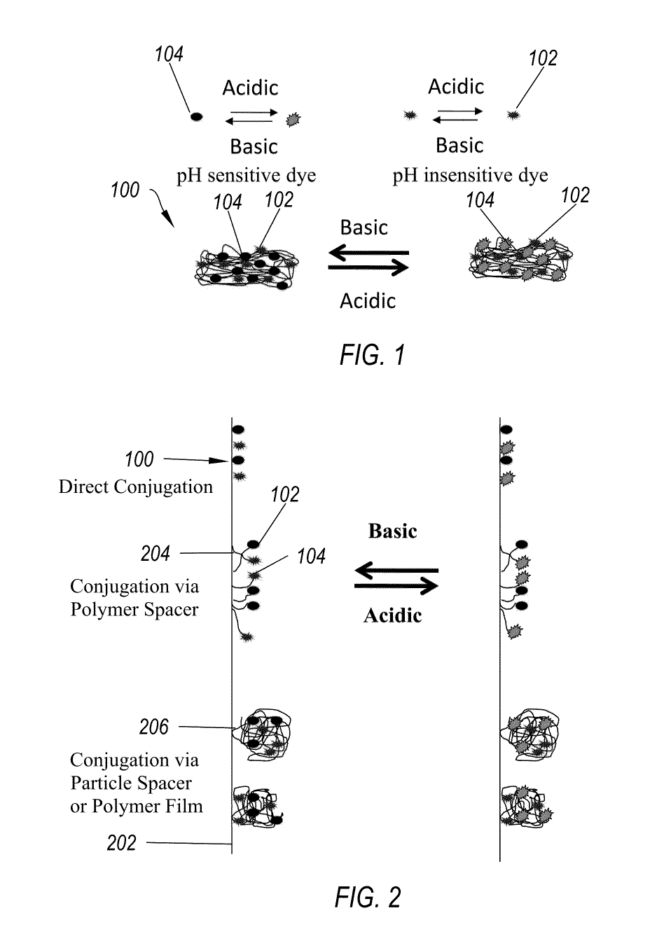

FIG. 1 illustrates an example of a pH detectable coating 100. The pH detectable coating 100 can be linked to medical implants and devices. This pH detectable coating 100 will then stay with the implants or devices to provide real time measurements of pH changes in the desired environment. For example, if the medical implant is placed within a patient's body, the pH of the body near the implant can be tracked in real time.

The pH detectable coating 100 can be placed on the surfaces of different devices and instruments. For example, the pH detectable coating 100 can be overlaid on top of medical devices. The pH detectable coating 100 can then provide pH information at the interface between the implants and host tissues. Additionally or alternatively, the pH detectable coating 100 can also be placed on the tip of medical instrument to detect pH changes in situ in vivo. Change of pH has been shown to affect protein structure and activities. Additionally or alternatively, the pH detectable coating 100 can be placed in containers for detecting the protein activities during storage. The freshness of food also affects pH. Additionally or alternatively, the pH detectable coating 100 can also be placed on the food processing equipment for the monitoring of the food quality.

FIG. 1 shows that the pH detectable coating 100 can include a pH insensitive fluorescent dye 102. The pH insensitive fluorescent dye 102 can provide a reference fluorescent intensity. I.e., the pH insensitive fluorescent dye 102 will provide a known fluorescence regardless of the pH of the environment of the attached surface. Examples of pH-insensitive dye 102 can include:
Cy-7 (λₑₓ 750 nm, Lumiprobe)
Dylight 800 (λₑₓ 770 nm, Thermo Scientific)
IRDye®800 (λₑₓ 786 nm, Licor)
Alexa Fluor®790 (λₑₓ 784 nm, Invitrogen)
HiLyte Fluor™750 (λₑₓ 754 nm, AnaSpec)
Oyster®800 (λₑₓ 778 nm, Boca Scientific)
Rhodamine β isothiocyanate (λₑₓ 540 nm, Sigma-Aldrich)
Texas Red derivatives (λₑₓ 595 nm, Invitrogen)
Alexa Fluor 680 (λₑₓ 670 nm, Invitrogen)
DyLight 680 (λₑₓ 670 nm, Pierce)
Cy5.5 NHS ester (λₑₓ 670 nm, Lumiprobe)
Alexa Fluor 546 (λₑₓ 555 nm, Invitrogen)
DyLight 549 (λₑₓ 555 nm, Pierce)
Cy3 NIH ester (λₑₓ 555 nm, Lumiprobe)

FIG. 1 also shows that the pH detectable coating 100 can include a pH sensitive fluorescent dye 104. The pH sensitive fluorescent dye 104 can fluoresce at a rate that is proportional to the pH of the surrounding environment. I.e., as the surrounding environment becomes more acidic or more alkaline the pH sensitive fluorescent dye 104 can increase or decrease its fluorescence. Examples of pH sensitive fluorescent dye 104 can include:
Oregon Green® 514 Carboxylic Acid (λₑₓ 489 nm, Molecular Probes)
pHrodo™ Red, succinimidyl ester (λₑₓ 566 nm, Molecular Probes)
SNARF®-5F 5-(and-6)-Carboxylic Acid (λₑₓ 488 nm, Molecular Probes)
SNARF®-4F 5-(and-6)-Carboxylic Acid (Molecular Probes)
5-(and-6)-Carboxy SNARF®-1 (Molecular Probes)
5(6)-Carboxynaphthofluorescein (λₑₓ 598 nm, Molecular Probes)
7-Hydroxycoumarin-3-carboxylic acid (λₑₓ 342 nm; λₑₘ 447 nm, Aldrich)
5-(and-6)-Carboxynaphthofluorescein (λₑₓ 489 nm, Molecular Probes)
6-Carboxy-4′,5′-Dichloro-2′,7′-Dimethoxyfluorescein (λₑₓ 522 nm, Molecular Probes)
BCECF (_{λex} 490 nm, Molecular Probes)
CyPHER5E (_{λex} 655 nm, GE Life Science)
HCyC-647 (λₑₓ 647 nm) (Hilderbrand et al., 2008)
Square-650-pH (K8-1407, SETA BioMedicals (Urbana, Ill., USA))

One of skill in the art will appreciate that any combination of pH insensitive fluorescent dye 102 and pH sensitive fluorescent dye 104 can be selected and used in the pH detectable coating 100 as long as their emission wavelengths do not overlap. I.e., the pH insensitive fluorescent dye 102 and pH sensitive fluorescent dye 104 should fluoresce at different wavelengths. The excitation and emission wavelengths of the pH insensitive fluorescent dye 102 and the pH sensitive fluorescent dye 104 can be in either visible or near-infrared light ranges, although near-infrared dyes are most suitable for in vivo imaging.

FIG. 2 illustrates various methods of attaching the pH detectable coating 100 to a surface 202. The method of attachment is based on convenience. I.e., whichever method allows for the easiest attachment can be used as long as the method of attachment does not affect the fluorescence sensitivity of the pH sensitive fluorescent dye 104. I.e., as long as the fluorescent intensity of the pH sensitive fluorescent dye 104 is not changed or changed in a known or measurable way.

FIG. 2 shows that the pH detectable coating 100 can be attached via direct conjugation. I.e., both the pH insensitive fluorescent dye 102 and the pH sensitive fluorescent dye 104 can be directly attached to the desired surface 202. For example, both the pH insensitive fluorescent dye 102 and the pH sensitive fluorescent dye 104 can be conjugated to the surface 202 via specific functional groups, such as carboxyl and amine groups or in any other desired manner.

FIG. 2 also shows that the pH detectable coating 100 can be attached via a polymer spacer 204. I.e., a polymer can be used as a spacer to attach both the pH-insensitive dye 102 and the pH-sensitive dye 104 to the surface 202. That is, the polymer spacer 204 is directly attached to the surface 202 and to both the pH-insensitive dye 102 and the pH-sensitive dye 104. Suitable polymers of the present invention include copolymers of water soluble polymers, including, but not limited to, dextran, derivatives of poly-methacrylamide, PEG, maleic acid, malic acid, and maleic acid anhydride and may include these polymers and a suitable coupling agent, including 1-ethyl-3 (3-dimethylaminopropyl)-carbodiimide, also referred to as carbodiimide. Polymers may be degradable or nondegradable or of a polyelectrolyte material. Degradable polymer materials include poly-L-glycolic acid (PLGA), poly-DL-glycolic, poly-L-lactic acid (PLLA), PLLA-PLGA copolymers, poly(DL-lactide)-block-methoxy polyethylene glycol, polycaprolacton, poly(caprolacton)-block-methoxy polyethylene glycol (PCL-MePEG), poly(DL-lactide-co-caprolactone)-block-methoxy polyethylene glycol (PDLLACL-MePEG), some polysaccharide (e.g., hyaluronic acid, polyglycan, chitoson), proteins (e.g., fibrinogen, albumin, collagen, extracellular matrix), peptides (e.g., RGD, polyhistidine), nucleic acids (e.g., RNA, DNA, single or double stranded), viruses, bacteria, cells and cell fragments, organic or carbon-containing materials, as examples. Nondegradable materials include natural or synthetic polymeric materials (e.g., polystyrene, polypropylene, polyethylene teraphthalate, polyether urethane, polyvinyl chloride, silica, polydimethyl siloxane, acrylates, arcylamides, poly (vinylpyridine), polyacroleine, polyglutaraldehyde), some polysaccharides (e.g., hydroxypropyl cellulose, cellulose derivatives, Dextran®, dextrose, sucrose, Ficoll®, Percoll®, arabinogalactan, starch), and hydrogels (e.g., polyethylene glycol, ethylene vinyl acetate, N-isopropylacrylamide, polyamine, polyethyleneimine, poly-aluminum chloride).

FIG. 2 further shows that the pH detectable coating 100 can be attached via a particle spacer or polymer film 206. I.e., a particle spacer or polymer film 206 can be used as a spacer to attach both the pH-insensitive dye 102 and the pH-sensitive dye 104 to the surface 202. That is, the particle spacer or polymer film 206 is directly attached to the surface 202 and to both the pH-insensitive dye 102 and the pH-sensitive dye 104. Particle spacer or polymer film 206 of the present invention may be applied to the surface of the instrument and device by methods known to one of the ordinary skill in the art, including by physical adsorption or chemical conjugation. The techniques described in accordance with the present invention may be used in vivo and in vitro. For example, nanoparticles can be used for coating blood bags, blood tubes, and food process containers. Particle spacer or polymer film of the present invention are generally provided as a metal particle, carbon particle, inorganic chemical particle, organic chemical particle, ceramic particle, graphite particle, polymer particle, protein particle, peptide particle, DNA particle, RNA particle, bacteria/virus particle, hydrogel particle, liquid particle or porous particle. Thus, the particles may be, for example, metal, carbon, graphite, polymer, protein, peptide, DNA/RNA, microorganisms (bacteria and viruses) and polyelectrolyte, and may be loaded with a light or color absorbing dye, an isotope, a radioactive species, a tag, or be porous having gas-filled pores. As used herein, the term “hydrogel” refers to a solution of polymers, sometimes referred to as a sol, converted into gel state by small ions or polymers of the opposite charge or by chemical crosslinking. Suitable polymers of the present invention can also include polymer listed above which can be used as polymer spacers.

One of skill in the art will appreciate that the pH detectable coating 100 can be attached via any other desired method. For example, both the pH-insensitive dye 102 and the pH-sensitive dye 104 to the surface 202 can be attached to the surface 202 via encapsulation, absorption, adsorption, covalent linkage, or any other desired attachment mechanism.

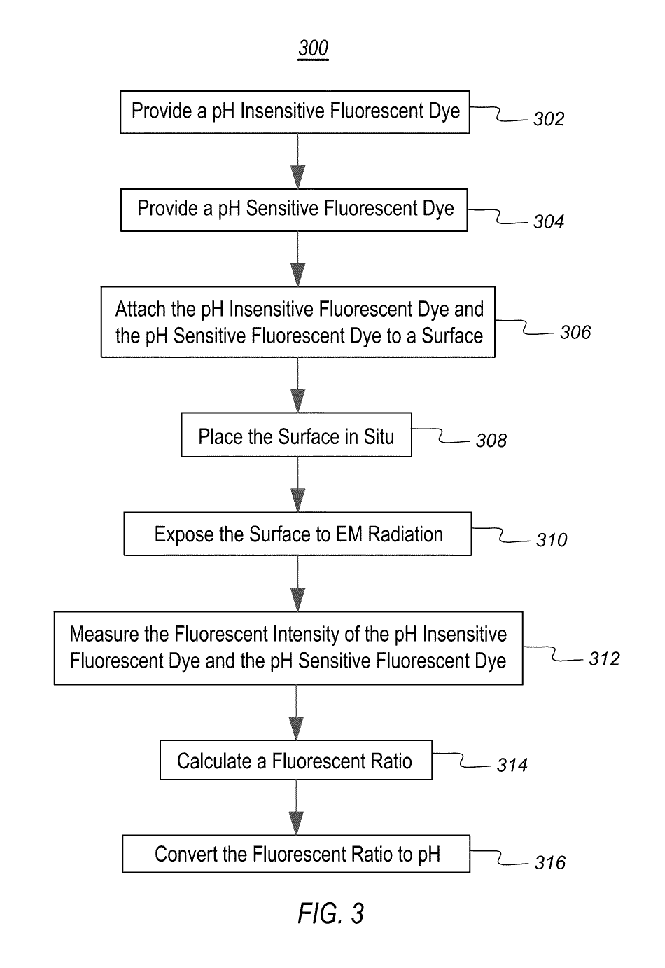

FIG. 3 is a flow chart illustrating a method 300 of detecting in situ pH. In at least one implementation, the method can be accomplished using a pH detectable coating, such as the pH detectable coating 100 of FIGS. 1-2. Therefore, the method 300 will be described, exemplarily, with reference to the pH detectable coating 100 of FIGS. 1-2. Nevertheless, one of skill in the art can appreciate that the method 300 can be used with a pH detectable coating other than the pH detectable coating 100 of FIGS. 1-2.

FIG. 3 shows that the method 300 can include providing 302 a pH insensitive fluorescent dye. The pH insensitive fluorescent dye can provide a reference fluorescent intensity. I.e., the pH insensitive fluorescent dye will provide a known fluorescence regardless of the pH of the environment of the medical device/implant.

FIG. 3 also shows that the method 300 can include providing 304 a pH sensitive fluorescent dye. The pH sensitive fluorescent dye can fluoresce at a rate that is proportional to the pH of the surrounding environment. I.e., as the surrounding environment becomes more acidic or more alkaline the pH sensitive fluorescent dye can increase its fluorescence.

FIG. 3 further shows that the method 300 can include attaching the pH insensitive fluorescent dye and the pH sensitive fluorescent dye to a surface. The method of attachment can be based on convenience. I.e., whichever method allows for the easiest attachment can be used as long as the method of attachment does not affect the fluorescence sensitivity of the pH sensitive fluorescent dye. I.e., as long as the fluorescence of the pH sensitive fluorescent dye is not changed or changed in a known or measurable way. For example, the attachment can include direct conjugation, conjugation via polymer spacer, conjugation via particle spacer or any other desired method.

FIG. 3 additionally shows that the method 300 can include placing 308 the surface in situ. I.e., the surface can be part of a medical device or implant which is placed 308 in a patient. Additionally or alternatively, the surface can be placed 308 in some other desired environment, as described above. For example, the surface can be placed 308 in a liquid for which pH monitoring is desired.

FIG. 3 moreover shows that the method 300 can include exposing 310 the surface to EM (electromagnetic) radiation. In particular, the surface is exposed 310 to EM radiation which will cause the pH insensitive and pH sensitive fluorescent dye to fluoresce. Fluorescence is the emission of light by a substance that has absorbed light or other electromagnetic radiation. It is a form of luminescence. In most cases, the emitted light has a longer wavelength, and therefore lower energy, than the absorbed radiation.

FIG. 3 also shows that the method 300 can include measuring 312 the fluorescent intensity of the pH insensitive fluorescent dye and the pH sensitive fluorescent dye. One of skill in the art will appreciate that the fluorescent intensity of the pH insensitive fluorescent dye can be measured 312 once or only at limited times. I.e., because the fluorescent intensity of the pH insensitive fluorescent dye is desired to remain relatively constant, it can be measured 312 continuously or at specified times. One of skill in the art will further appreciate that the fluorescent intensity of the pH sensitive fluorescent dye can be measured 312 in real time to monitor pH changes.

FIG. 3 further shows that the method 300 can include calculating 314 a fluorescent ratio. The fluorescent ratio is the ratio of the fluorescent intensity of the pH insensitive fluorescent dye relative to the fluorescent intensity of the pH sensitive fluorescent dye. I.e. the fluorescent ratio can be calculated 314 using equation 1 or another similar equation.

$$FR = \frac{{FI}_{pH sensitive dye} }{{FI}_{pH insensitive dye} } \qquad Equation 1$$

where FR is the fluorescent ratio, FI_{pH sensitive fluorescent dye} is the fluorescent intensity of the pH sensitive fluorescent dye and FI_{pH insensitive fluorescent dye} is the fluorescent intensity of the pH insensitive fluorescent dye.

FIG. 3 additionally shows that the method 300 can include converting 316 the fluorescent ratio to pH. In particular, converting 316 the fluorescent ratio to pH can include using an equation that is calculated through observations with known pH values. I.e., the fluorescent ratio can be measured at known pH values in order to create an equation which can be used to covert 316 fluorescent ratio to pH. Additionally or alternatively, the fluorescent ratio can be used to normalize the fluorescent intensity of the pH sensitive fluorescent dye, which is then converted 316 to pH.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

### Examples of Use and Results

To prove the concept, CypHer5E was used as a pH-sensitive cyanine dye which has minimal fluorescence at neutral pH but becomes highly fluorescent with an emission peak at ˜670 nm in an acidic environment. Oyster®800, which has a constant fluorescence with an emission peak at ˜794 nm, was used as a pH-insensitive dye. The pH sensors were fabricated by conjugating both dyes into coating made of poly(N-isopropylacrylamide) (PNIAPM) particles. PNIPAM particles were prepared by using the precipitation polymerization method. The material was lyophilized and stored at 4-8° C. for further use. Average size, size distribution and zeta potential of PNIPAM particles were measured using dynamic light scattering (Zeta PALS, Brookhaven Instruments Corp., NY). PNIPAM spheres (4 mg/ml) were suspended in sterilized PBS/0.5M sodium carbonate solution (pH8.3) and then mixed with CypHer5E dye (1 mg/ml). Following overnight reaction at room temperature, the CypHer5E-labled PNIPAM spheres were dialyzed against sterilized DI water, lyophilized and then re-suspended in 10 ml PBS (pH7.4). Oyster®800 dye (0.05 mg/ml) was added and reacted with CypHer5E-labled PNIPAM spheres in the dark at room temperature. Following dialysis with DI water in the dark, PNIPAM-CypHer5E-Oyster®800 pH sensors were lyophilized and stored at 4-8° C. The particle-based sensors possess sufficient residual functional groups which can be conjugated to the device surfaces.

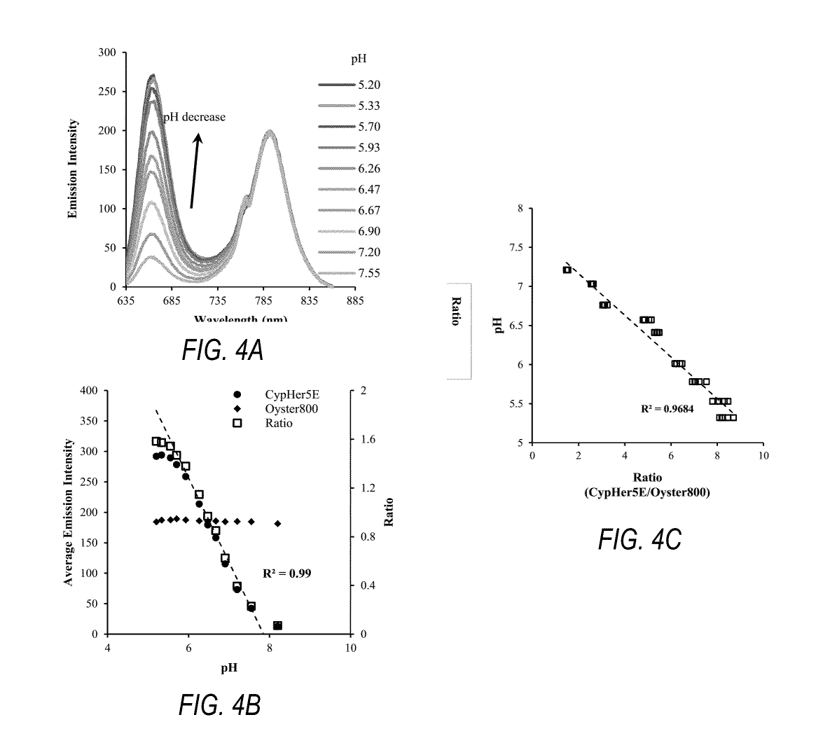

These dye-conjugated pH sensor coatings were subsequently tested for their pH sensitivities in vitro. By scanning the fluorescence spectrum of the probes at pH between 5.20 and 7.55, two distinct and separate peaks were found. The lower-wavelength peak shared an identical spectrum with CypHer5E (maximal at ˜670 nm) and the higher-wavelength peak derives from Oyster®800 (maximal at ˜794 nm) (FIG. 4A). By varying pH from 4.4 to 9.0, the average fluorescence intensities from 670 nm to 730 nm (CypHer5E) were substantially increased whereas the average fluorescence intensities from 800 nm to 860 nm (Oyster®800) were unchanged throughout the pH range (the wavelengths were chosen in order to compare the results from the KODAK in vivo machine) (FIG. 4B). The ratios of the average fluorescence intensities between CypHer5E (pH-sensitive dye) and Oyster®800 (pH-insensitive dye) were also calculated to provide quantitative measurement of pH in an aqueous environment. The ratios of average fluorescence intensities had a strong correlation with pH values from 5.53 to 7.55 (Ratio=−0.692*pH+5.439, R²=0.99). These two wavelength ranges were used to generate the quantitative data in the following studies. The pH sensors were subsequently tested for their ratiometric imaging capability. Using pH sensor coating to measure various pH levels in solution, we observed dramatic changes in pH ratiometric imaging in vitro correlating with change of pH from 4.40 to 9.00. There was an almost linear relationship between the florescence intensity ratios and pH values from 5.78 to 7.65 (Ratio=−3.629*pH+28.103, R²=0.97) (FIG. 4C).

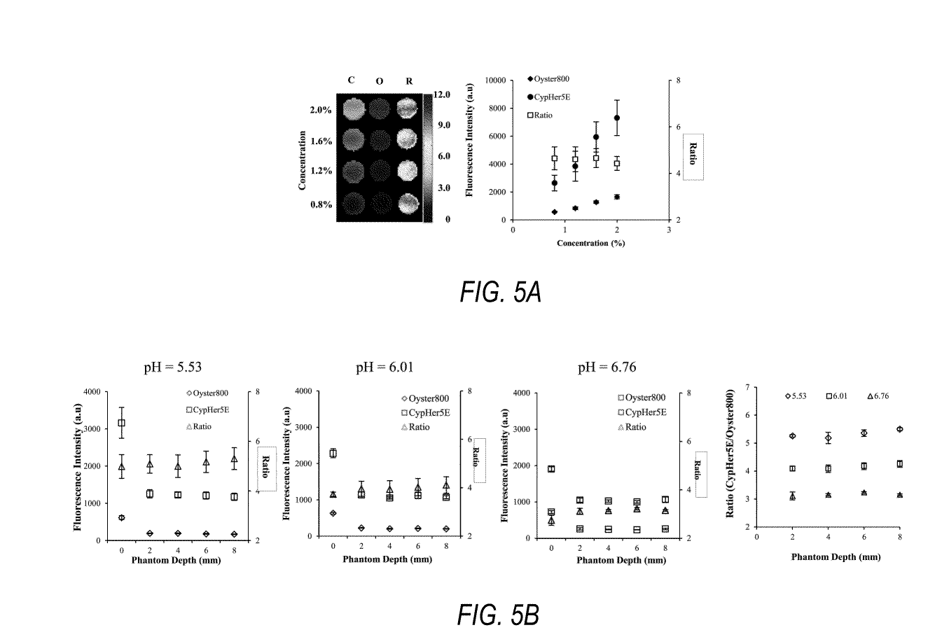

Subsequent studies were carried out to detect the effect of probe concentrations and skin thickness on the accuracy of pH detection by pH-sensitive coating. By adding different concentrations (0.8-2.0% w/v) of the pH probes into pH 6.76 solutions, an increase in fluorescent intensities at both wavelengths was observed (FIG. 5A). However, the ratios of both fluorescent peaks were nearly constant at ˜4.58 fold, independent of the sensor concentrations in vitro (FIG. 5B). Next, whether the ratio of CypHer5E and Oyster®800 fluorescence could correctly measure the pH at different depths in skin phantoms was assessed. For that, different thicknesses (2-8 mm) of the skin phantoms were placed between the same amount (1% w/v) of pH sensors and the light source. Indeed, the fluorescence intensities decreased with increasing skin phantom thickness. Nevertheless, the ratio of both fluorescent intensities at different pH values was nearly constant regardless of skin phantom thickness (2 to 8 mm) (FIG. 5B).

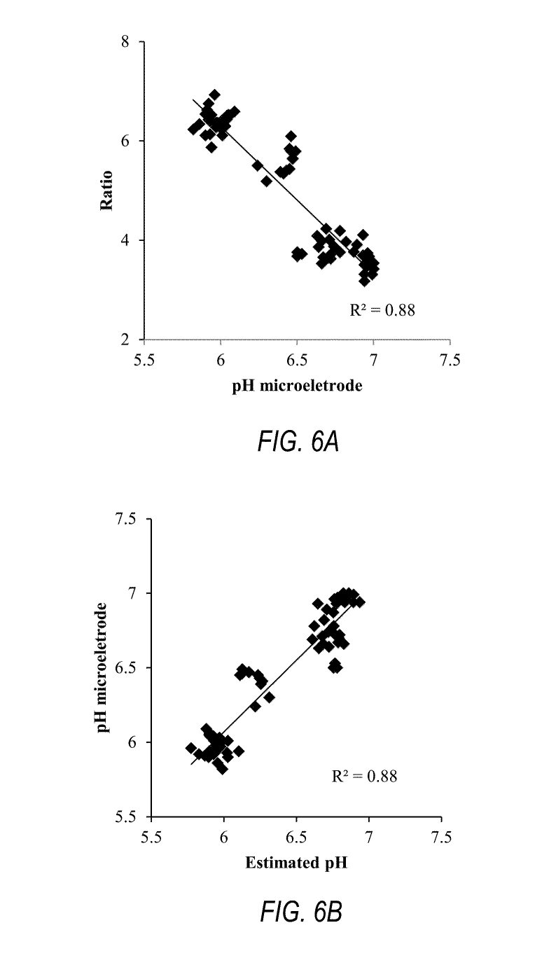

Additional experiments were carried out to assess the accuracy of the pH-sensitive coatings. For that, ratiometric imaging techniques were used to detect the solution pH via pH-sensitive coatings. Simultaneously, the pH values were obtained using a glass microelectrode probe pH microelectrode (M1-431) connected to an Accumet pH meter. By comparing both sets of data, a good relationship was found (R²=0.88) between the pH levels measured by the microelectrode probe and the estimated pH from the ratiometric imaging (FIG. 6A). In addition, the correlation between pH estimated using the pH ratio and the measured pH using the microelectrode was determined and there is likewise a strong relationship (R²=0.88) (FIG. 6B).

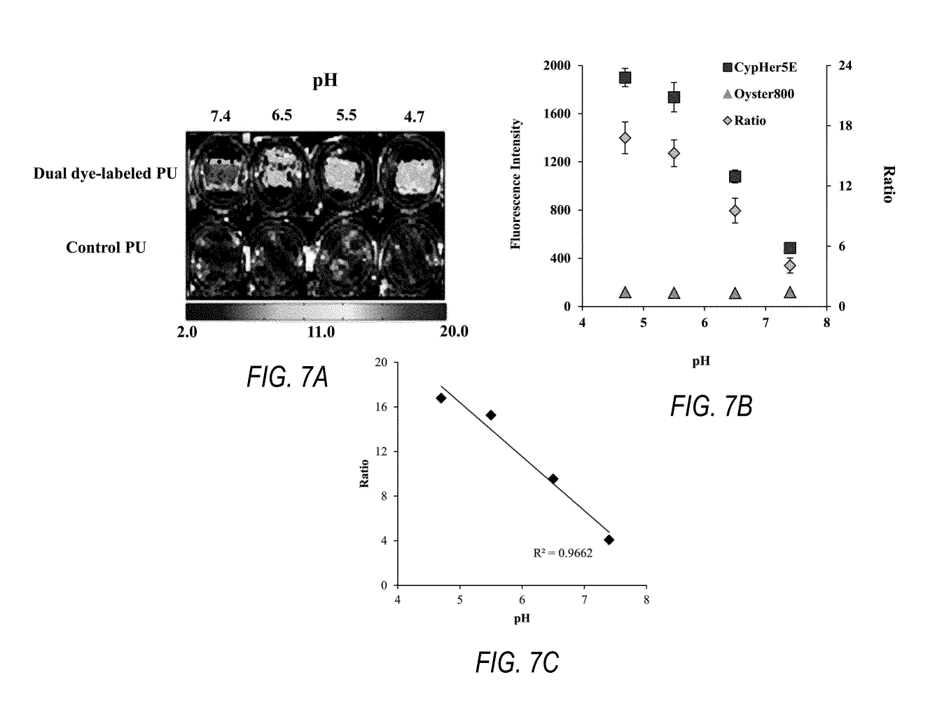

To test the efficacy of the coating to detect pH changes surrounding medical devices, polyurethane catheters coated with both CypHER5E and Oyster®800 dyes were used as an example. The dye-coated polyurethane catheters were prepared as follows: the surfaces of polyurethane catheters were first decorated with NH₂ groups using plasma glow discharge technique; and then CypHER5E and Oyster®800 dyes were sequentially introduced onto the surfaces of catheters via EDC coupling chemistry. By placing the dual dye-coated catheters in different pH solutions, the ratiometric images were taken on those catheters (FIG. 7A). At pH-sensitive dye wavelength, the fluorescent signals increased with reduced pH (FIG. 7B). On the other hand, the fluorescent signals at the pH-insensitive dye wavelength range remain unchanged. Most importantly, there is a good relationship between pH changes and florescent ratio changes (FIG. 7C). These results support that the pH coating can enable us to detect pH changes surrounding medical devices.

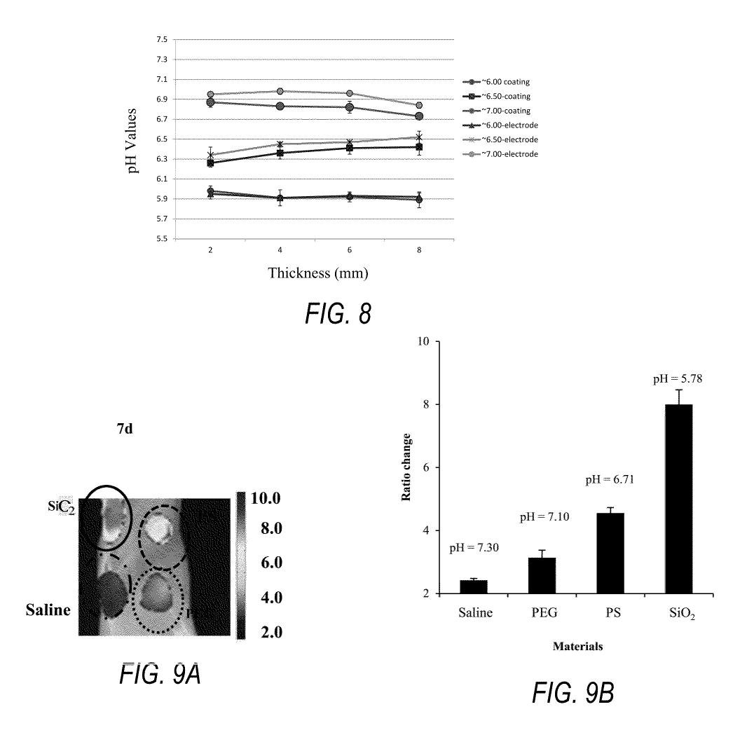

Further studies also test the ability of the coating to provide pH information at different depths. For that, polyurethane catheters coated with pH-sensitive coating were placed inside gelatin tissue phantom with different pH at different depths (2-8 mm). The ratiometric imaging results were also compared with the results obtained using a glass microelectrode probe pH microelectrode (M1-431) connected to an Accumet pH meter. pH coating was found to detect the pH values at different depths with very little variations (FIG. 8). The ratiometric imaging results were also very close to the pH values obtained using pH microelectrode.

The pH-sensitive coatings were tested for their ability to monitor pH changes using mouse implantation model. Different particles, including silicon dioxide particles (SiO₂), polystyrene particles (PS), polyethylene glycol particles (PEG), were coated with pH-sensitive polymer prior to subcutaneous implantation in Balb/C mice. After implantation for 7 days, the ratiometric images were taken at the whole animals (FIG. 9A). For imaging analyses, animals were anesthetized by isoflurane inhalation and then imaged by the KODAK In vivo FX Pro (Kodak, USA). Mice were imaged for the CypHer5E and Oyster®800 channels at the specified time points. The ratiometric images of CypHer5E/Oyster®800 were calculated after background correction. Polygon regions of interest (ROI) were drawn over the inflammatory locations in the ratiometric imaging. The mean intensities for all pixels in the pH ratiometric imaging were calculated. The ratio data were then converted into estimated pH values for in vivo calibrations. The mean ratiometric change for all implantation sites was measured (FIG. 9B). All four materials triggered varying ratiometric changes in the order: SiO₂>PS>PEG>saline. Specifically, the SiO₂ implantation site has a pH of 5.8±0.1, polystyrene has a pH of 6.7±0.1, PEG have a pH of 7.1±0.1 and the saline-injected control tissue has a pH of 7.3±0.1 (FIG. 9B).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A pH detectable coating, the pH detectable coating comprising:
a pH insensitive fluorescent dye;
a pH sensitive fluorescent dye;
wherein the pH insensitive fluorescent dye and the pH sensitive fluorescent dye are attached to a surface; and
wherein the ratio of the fluorescent intensity of the pH insensitive fluorescent dye to the fluorescent intensity of the pH sensitive fluorescent dye varies according to the pH of an environment into which the surface is placed.

2. The pH detectable coating of claim 1, wherein the surface includes the surface of a medical device.

3. The pH detectable coating of claim 1, wherein the surface includes the surface of a medical implant.

4. The pH detectable coating of claim 1, wherein the surface includes the surface of a food processing equipment.

5. The pH detectable coating of claim 1, wherein the surface includes the inner surface of a container.

6. The pH detectable coating of claim 1, wherein the emission wavelengths of the pH insensitive fluorescent dye and the pH sensitive fluorescent dye do not overlap.

7. The pH detectable coating of claim 1, wherein the pH insensitive fluorescent dye includes at least one of:
Cy-7;
Dylight 800;
IRDye^{@}800;
Alexa Fluor^{@}790;
HiLyte Fluor™750;
Oyster^{@}800;
Rhodamine β isothiocyanate (λₑₓ 540 nm, Sigma-Aldrich);
Texas Red derivatives (λₑₓ 595 nm, Invitrogen);
Alexa Fluor 680 (λₑₓ 670 nm, Invitrogen);
DyLight 680 (λₑₓ 670 nm, Pierce);
Cy5.5 NHS ester (λₑₓ 670 nm, Lumiprobe);
Alexa Fluor 546 (λₑₓ 555 nm, Invitrogen);
DyLight 549 (λₑₓ 555 nm, Pierce); or
Cy3 NIH ester (λₑₓ 555 nm, Lumiprobe).

8. The pH detectable coating of claim 1, wherein the pH sensitive fluorescent dye includes at least one of:
Oregon Green® 514 Carboxylic Acid;
pHrodo™ Red, succinimidyl ester;
SNARF®-5F 5-(and-6)-Carboxylic Acid;
SNARF®-4F 5-(and-6)-Carboxylic Acid;
5-(and-6)-Carboxy SNARF®-1;
5(6)-Carboxynaphthofluorescein;
7-Hydroxycoumarin-3-carboxylic acid;
5-(and-6)-Carboxynaphthofluorescein;
6-Carboxy-4′,5′-Dichloro-2′,7′-Dimethoxyfluorescein;
BCECF;
CyPHER5E;
HCyC-647; or
Square-650-pH.

9. The pH detectable coating of claim 1, wherein the emission wavelength of the pH insensitive fluorescent dye is in the visible light range.

10. The pH detectable coating of claim 1, wherein the emission wavelength of the pH insensitive fluorescent dye is in the near-infrared range.

11. The pH detectable coating of claim 1, wherein the emission wavelength of the pH sensitive fluorescent dye is in the visible light range.

12. The pH detectable coating of claim 1, wherein the emission wavelength of the pH sensitive fluorescent dye is in the near-infrared range.

13. A method of manufacturing a pH detectable coating, the method comprising:
attaching a pH insensitive fluorescent dye to a surface; and
attaching a pH sensitive fluorescent dye to the surface;
wherein the ratio of the fluorescent intensity of the pH insensitive fluorescent dye to the fluorescent intensity of the pH sensitive fluorescent dye varies according to the pH of the environment into which the surface is placed.

14. The method of claim 13, wherein attaching the pH insensitive fluorescent dye to the surface includes direct conjugation.

15. The method of claim 13, wherein attaching the pH insensitive fluorescent dye to the surface includes attachment via a polymer spacer.

16. The method of claim 13, wherein attaching the pH insensitive fluorescent dye to the surface includes attachment via a particle spacer.

17. A method of measuring pH in situ, the method comprising:
providing a pH detectable coating on a surface, the pH detectable coating including:
a pH insensitive fluorescent dye; and
a pH sensitive fluorescent dye;
wherein the ratio of the fluorescent intensity of the pH insensitive fluorescent dye to the fluorescent intensity of the pH sensitive fluorescent dye varies according to the pH of an environment into which the surface is placed;
placing the surface in an environment;
exposing the surface to electromagnetic radiation; and
measuring a fluorescent ratio; and
converting the fluorescent ratio to a pH value.

18. The method of claim 17, wherein measuring the fluorescent ratio includes:
measuring the fluorescent intensity of the pH insensitive fluorescent dye; and
measuring the fluorescent intensity of the pH sensitive fluorescent dye.

19. The method of claim 17, wherein converting the fluorescent ratio to a pH value includes comparing the fluorescent ratio to observations of the surface placed in a second environment of known pH.
